(19) United States
(12) Patent Application Publication
(10) Pub. No.: US 2023/0178214 A1
(43) Pub. Date: Jun. 8, 2023

(54) SYSTEMS AND METHODS FOR CREATING CONTEXT-BASED PERSONALIZED NUTRITION PLANS AND RECOMMENDATIONS

(71) Applicant: REVIV GLOBAL LTD, Knutsford (GB)

(72) Inventors: Sarah Lomas, Cheshire (GB); Michael Barnish, Cheshire (GB); Johnny Parvani, Phoenix, AZ (US); Jack Worne, Cheshire (GB)

(21) Appl. No.: 18/102,432

(22) Filed: Jan. 27, 2023

Related U.S. Application Data
(63) Continuation-in-part of application No. 17/876,352, filed on Jul. 28, 2022.
Application No. 17/876,352 is a continuation-in-part of application No. 17/876,383, filed on Jul. 28, 2022, now Pat. No. 11,600,375.
Application No. 17/876,383 is a continuation-in-part of application No. 17/880,920, filed on Aug. 4, 2022.
(60) Provisional application No. 63/227,943, filed on Jul. 30, 2021.
(60) Provisional application No. 63/227,952, filed on Jul. 30, 2021.
(60) Provisional application No. 63/230,516, filed on Aug. 6, 2021.

(51) Int. Cl. G16H 20/60; G16H 10/60

(52) U.S. Cl. CPC G16H 20/60; G16H 10/60

(57) ABSTRACT

A method of creating a context-based personalized nutrition recommendation for reducing risk of disease includes receiving genetic information, medical information, therapeutic objectives, and lifestyle information of an individual. A location of the individual is determined using a position sensor and data associated with the location of the individual, including real-time air quality data, is retrieved. Fitness data of the individual is collected using a wearable device. One or more micronutrients for the individual are identified based on the genetic information, medical information, therapeutic objectives, lifestyle information, and fitness data of the individual and based on the real-time air quality data. Food, an oral supplement, a topical treatment, an inhaled nutrition treatment, an intravenous nutrition therapy formula, and an intramuscular nutrition therapy formula that provide the identified one or more micronutrients are identified, and one of these is recommended based on a cost and an effectiveness of each of them.

### RELATED APPLICATIONS

This application is a continuation-in-part of U.S. Utility application Ser. No. 17/876,352, filed Jul. 28, 2022, which claims the benefit of U.S. Provisional Application No. 63/227,943, filed Jul. 30, 2021, the disclosures of which are hereby incorporated by reference in their entirety. This application is also a continuation-in-part of U.S. Utility application Ser. No. 17/876,383, filed Jul. 28, 2022, which claims the benefit of U.S. Provisional Application No. 63/227,952, filed Jul. 30, 2021, the disclosures of which are hereby incorporated by reference in their entirety. This application is also a continuation-in-part of U.S. Utility application Ser. No. 17/880,920, filed Aug. 4, 2022, which claims the benefit of U.S. Provisional Application No. 63/230,516, filed Aug. 6, 2021, the disclosures of which are hereby incorporated by reference in their entirety.

### TECHNICAL FIELD

This disclosure relates generally to nutrition plans and recommendations, and more specifically to systems and methods that create context-based personalized nutrition plans and recommendations, accounting for an individual's genetic and other medical information, lifestyle, environment, and other contextual information.

### BACKGROUND

Achieving and maintaining optimal health and proper function of the human body requires proper nutrition and hydration, including a correct balance of nutrients such as macronutrients (e.g., proteins, carbs, fats) and micronutrients (e.g., vitamins, minerals, antioxidants). Maintaining nutrient intake to equal an individual's nutrient demand supports normal body physiology and thus helps prevent disease. However, the modern approach to medicine is more of a reactive approach to health, in which intervention only occurs when the disruption to normal body physiology is advanced enough to produce a symptom. Indeed, good nutrition is often sacrificed for convenience. As a result, the prevalence of chronic disease is rapidly increasing. Even with global research efforts and new drugs, such disease still has major impacts on individuals and societies.

Further complicating these problems, each individual is unique, with different genetic makeups, lifestyles, cultures, environments, and other circumstances. Thus, the nutrient demand for one person will not necessarily be the same as another person, and what is beneficial to one person may be less useful, or even detrimental, to another. Yet most individuals do not know how their unique circumstances (e.g., their genetic makeup, their culture, their environment, etc.) should affect their nutrition and other health choices. Moreover, while there are various avenues for obtaining nutrients (such as food intake, oral supplements, topical supplements or treatments, intravenous (“IV”) nutrition therapy, and intramuscular (“TM”) nutrition therapy), the availability of each avenue or options within each avenue may differ for different individuals due to location, culture, economic circumstances, or other factors.

### SUMMARY

Aspects of this disclosure relate to a method of creating a context-based personalized nutrition recommendation. In some embodiments, the method includes receiving genetic information, medical information, and therapeutic objectives of an individual. In some embodiments, the genetic information is determined by a genetic test. In some embodiments, the method includes receiving lifestyle information about the individual. In some embodiments, the lifestyle information includes information about physical activities, mental activities, occupation, social network, financial security, culture, habits, and preferences of the individual. In some embodiments, the method includes using a position sensor to determine a location of the individual. In some embodiments, the method includes retrieving data associated with the location of the individual from the position sensor. In some embodiments, the data comprises real-time air quality data. In some embodiments, the method includes collecting fitness data of the individual using a wearable device. In some embodiments, the method includes identifying one or more micronutrients for the individual based on the genetic information, medical information, therapeutic objectives, lifestyle information, and fitness data of the individual and based on the real-time air quality data associated with the location of the individual. In some embodiments, the method includes identifying at least two of a food, an oral supplement, a topical treatment, inhaled nutrition treatment, an intravenous nutrition therapy formula, and an intramuscular nutrition therapy formula that provide the identified one or more micronutrients, and recommending one of the identified at least two of the food, the oral supplement, the topical treatment, the inhaled nutrition treatment, the intravenous nutrition therapy formula, and the intramuscular nutrition therapy formula based on a cost of each of the identified at least two of the food, the oral supplement, the topical treatment, the inhaled nutrition treatment, the intravenous nutrition therapy formula, and the intramuscular nutrition therapy formula, the financial security of the individual, and an effectiveness of each of the at least two of the food, the oral supplement, the topical treatment, the inhaled nutrition treatment, the intravenous nutrition therapy formula, and the intramuscular nutrition therapy formula in providing the identified one or more micronutrients.

Particular implementations may comprise one or more of the following features. In some embodiments, the recommending is performed by a first application on an electronic device, and receiving lifestyle information includes retrieving data associated with a second application on the electronic device. In some embodiments, the data associated with the location of the individual comprises available options of food, oral supplements, topical treatments, inhaled nutrition treatments, intravenous nutrition therapy formulas, and intramuscular nutrition therapy formulas at the location of the individual. In some embodiments, the method also includes receiving a recommendation request from the individual. In some embodiments, the method also includes developing a nutrition plan based on the genetic information, medical information, therapeutic objectives, lifestyle information, and fitness data of the individual. In some embodiments, the method also includes adjusting the nutrition plan based on the real-time air quality data associated with the location of the individual. In some embodiments, the method also includes adjusting the nutrition plan based on updated medical information, therapeutic objectives, or lifestyle information of the individual.

Aspects of this disclosure relate to a system for creating a context-based personalized nutrition recommendation. In some embodiments, the system includes a genetic test configured to determine genetic information of an individual. In some embodiments, the system includes an electronic device configured to receive the genetic information of the individual, as well as medical information, therapeutic objectives, and lifestyle information of the individual. In some embodiments, the system includes a plurality of sensors in communication with the electronic device. In some embodiments, the plurality of sensors includes a position sensor, an accelerometer, and a heart rate monitor. In some embodiments, the electronic device is configured to determine real-time air quality data based on location data from the position sensor. In some embodiments, the electronic device is configured to determine fitness data of the individual based on motion data from the accelerometer and heart rate data from the heart rate monitor. In some embodiments, the system includes a server that includes a food database, an oral supplement database, a topical treatment database, an inhaled nutrition therapy database, an intravenous nutrition therapy database, an intramuscular nutrition therapy database, and a mapping of genetic information, medical information, therapeutic objectives, lifestyle information, air quality data, and fitness data to one or more micronutrients. In some embodiments, the server is in communication with the electronic device. In some embodiments, the system is configured to use the mapping to identify one or more micronutrients for the individual based on the genetic information, medical information, therapeutic objectives, lifestyle information, and fitness data of the individual and based on the real-time air quality data. In some embodiments, the system is configured to identify at least two of a food from the food database, an oral supplement from the oral supplement database, a topical treatment from the topical treatment database, an inhaled nutrition treatment from an inhaled nutrition treatment database, an intravenous nutrition therapy formula from the intravenous nutrition therapy database, and an intramuscular nutrition therapy formula from the intramuscular nutrition therapy database that provide the identified one or more micronutrients. In some embodiments, the system is configured to recommend one of the at least two of the food, the oral supplement, the topical treatment, the inhaled nutrition treatment, the intravenous nutrition therapy formula, and the intramuscular nutrition therapy formula based on a cost and an effectiveness of each of the food, the oral supplement, the topical treatment, the inhaled nutrition treatment, the intravenous nutrition therapy formula, and the intramuscular nutrition therapy formula.

Particular implementations may comprise one or more of the following features. In some embodiments, the server includes multiple servers. In some embodiments, at least one of the plurality of sensors is disposed within the electronic device. In some embodiments, at least one of the plurality of sensors is disposed within a wearable device in communication with the electronic device. In some embodiments, the electronic device is configured to receive a recommendation request from the individual. In some embodiments, the system is configured to develop a nutrition plan based on the genetic information, medical information, therapeutic objectives, lifestyle information, and fitness data of the individual. In some embodiments, the electronic device is a mobile device.

Aspects of this disclosure relate to a method of creating a context-based personalized nutrition recommendation. In some embodiments, the method includes determining genetic information of an individual with a genetic test. In some embodiments, the method includes receiving medical information, therapeutic objectives, and lifestyle information of the individual. In some embodiments, the method includes mapping the genetic information, medical information, therapeutic objectives, and lifestyle information of the individual to one or more micronutrients. In some embodiments, the method includes identifying at least one of a food, an oral supplement, a topical treatment, an inhaled nutrition treatment, an intravenous nutrition therapy formula, and an intramuscular nutrition therapy formula that each provide the one or more micronutrients. In some embodiments, the method includes recommending the at least one of the food, the oral supplement, the topical treatment, the inhaled nutrition treatment, the intravenous nutrition therapy formula, and the intramuscular nutrition therapy formula based on a cost and an effectiveness of each of the at least one of the food, the oral supplement, the topical treatment, the inhaled nutrition treatment, the intravenous nutrition therapy formula, and the intramuscular nutrition therapy formula. In some embodiments, the method includes (after the individual has received the one or more micronutrients via the recommended at least one of the food, the oral supplement, the topical treatment, the inhaled nutrition treatment, the intravenous nutrition therapy formula, and the intramuscular nutrition therapy formula) receiving feedback on an effectiveness of the recommended at least one of the food, the oral supplement, the topical treatment, the inhaled nutrition treatment, the intravenous nutrition therapy formula, and the intramuscular nutrition therapy formula. In some embodiments, the method includes creating future recommendations for the individual and for other individuals based on the feedback.

Particular implementations may comprise one or more of the following features. In some embodiments, the lifestyle information includes information about physical activities, mental activities, occupation, social network, financial security, culture, habits, and preferences of the individual. In some embodiments, the method also includes receiving a recommendation request from the individual. In some embodiments, the method also includes developing a nutrition plan based on the genetic information, medical information, therapeutic objectives, lifestyle information, and fitness data of the individual. In some embodiments, the method also includes adjusting the nutrition plan based on the real-time air quality data associated with the location of the individual. In some embodiments, the method also includes adjusting the nutrition plan based on updated medical information, therapeutic objectives, or lifestyle information of the individual.

The foregoing and other aspects, features, applications, and advantages will be apparent to those of ordinary skill in the art from the specification, drawings, and the claims. Unless specifically noted, it is intended that the words and phrases in the specification and the claims be given their plain, ordinary, and accustomed meaning to those of ordinary skill in the applicable arts. The inventors are fully aware that he can be his own lexicographer if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112(f). Moreover, even if the provisions of 35 U.S.C. § 112(f) are invoked to define the claimed aspects, it is intended that these aspects not be limited only to the specific structure, material or acts that are described in the preferred embodiments, but in addition, include any and all structures, materials or acts that perform the claimed function as described in alternative embodiments or forms of the disclosure, or that are well known present or later-developed, equivalent structures, material or acts for performing the claimed function.

The foregoing and other aspects, features, and advantages will be apparent to those of ordinary skill in the art from the specification, drawings, and the claims.

### BRIEF DESCRIPTION OF THE DRAWINGS

The disclosure will hereinafter be described in conjunction with the appended drawings.

FIG. 1 shows a system for creating context-based personalized nutrition plans and recommendations according to some embodiments.

FIG. 2 shows a system for creating context-based personalized nutrition plans and recommendations according to some embodiments.

FIG. 3 shows an electronic device for use in a system for creating context-based personalized nutrition plans and recommendations according to some embodiments.

FIG. 4 shows a schematic diagram of computing devices that can be used to perform or implement the embodiments disclosed herein.

### DETAILED DESCRIPTION

This disclosure, its aspects and implementations, are not limited to the specific components, methods, or other examples disclosed herein. Many additional components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

While this disclosure includes a number of implementations in many different forms, the drawings show particular implementations that will be described in detail with the understanding that the present disclosure is to be considered as an exemplification of the principles of the disclosed methods and systems and is not intended to limit the broad aspect of the disclosed concepts to the implementations illustrated.

Achieving and maintaining optimal health and proper function of the human body requires proper nutrition and hydration, including a correct balance of nutrients such as macronutrients (e.g., proteins, carbs, fats) and micronutrients (e.g., vitamins, minerals, antioxidants). Maintaining nutrient intake to equal an individual's nutrient demand supports normal body physiology and thus helps prevent disease. However, the modern approach to medicine is more of a reactive approach to health, in which intervention only occurs when the disruption to normal body physiology is advanced enough to produce a symptom. Indeed, good nutrition is often sacrificed for convenience. As a result, the prevalence of chronic disease is rapidly increasing. Even with global research efforts and new drugs, such disease still has major impacts on individuals and societies.

Further complicating these problems, each individual is unique, with different genetic makeups, lifestyles, cultures, environments, and other circumstances. Thus, the nutrient demand for one person will not necessarily be the same as another person, and what is beneficial to one person may be less useful, or even detrimental, to another. Yet most individuals do not know how their unique circumstances (e.g., their genetic makeup, their culture, their environment, etc.) should affect their nutrition and other health choices. Moreover, while there are various avenues for obtaining nutrients (such as food intake, oral supplements, topical supplements or treatments, intravenous (“IV”) nutrition therapy, and intramuscular (“TM”) nutrition therapy), the availability of each avenue or options within each avenue may differ for different individuals due to location, culture, economic circumstances, or other factors.

Improved systems and methods that account for an individual's unique circumstances are desirable. For example, a system that assists individuals in making nutrition, lifestyle, and health decisions based on their genetic data and other contextual information would reduce the impact of chronic and other diseases on both individuals and societies. The present disclosure describes systems and methods that receive contextual information about an individual (e.g., genetic information, medical information, therapeutic objectives, lifestyle information, habits, stresses, etc.). Because this contextual information determines a nutrient demand for each individual, the pieces of contextual information may collectively be considered as determinants of nutrition. The disclosed systems and methods use the received contextual information to identify micronutrients that the individual needs (either at a specific point in time or as part of an overall nutrition plan). Scientific literature and/or prior treatment results (collected from a number of users) may be used to map the contextual information to the right micronutrients for any specific situation. Over time, this mapping will become more accurate and more specific because the systems will be able to determine how each factor and combination of factors affects the nutrient demand of an individual.

In some embodiments, the disclosed systems and methods identify different routes or delivery methods of providing the identified micronutrients. These routes may include food, oral supplements, intravenous nutrition therapy formulas, intramuscular nutrition therapy formulas, or topical supplements. After identifying the options for delivering the identified micronutrients (e.g., which foods, which oral supplements, which nutrition therapy formulas, etc.), the systems may recommend the best option for the individual based on a number of factors, such as cost, effectiveness, availability in the individual's location, personal preference, etc. After an individual receives the micronutrients through the recommended route, feedback may be received regarding the effectiveness of the micronutrients and the recommended route. The feedback may be subjective (e.g., indications from the individual about how the individual felt, whether symptoms lessened, etc.) and/or objective (e.g., vital signs, blood tests, etc.). This feedback may be used to update a mapping between contextual information and micronutrients and/or routes of providing the micronutrients. Thus, as already noted above, over time the mapping becomes more accurate and more specific because the system develops a more complete picture as to how each factor and combination of factors affects the nutrient demand.

The systems and methods may be used in a variety of situations. For example, in some embodiments, the systems may be used to receive a specific, one-time recommendation, or they may be used to receive a long-term nutrition plan. As another example, the systems may proactively provide recommendations, or they may only provide recommendations in response to an inquiry from a user. The systems may also be used for specific settings, such as while grocery shopping, to determine an action to take. Moreover, the recommendations may be a nutrition recommendation, or they may be another health-related or lifestyle-related recommendation (e.g., to increase physical activity). In some embodiments, the systems and methods disclosed herein may use any or all of the features or principles disclosed in U.S. patent application Ser. No. 17/876,352, filed Jul. 28, 2022, U.S. patent application Ser. No. 17/876,383, filed Jul. 28, 2022, and U.S. patent application Ser. No. 17/880,920, filed Aug. 4, 2022, the disclosures of each of which are incorporated herein by reference in their entireties.

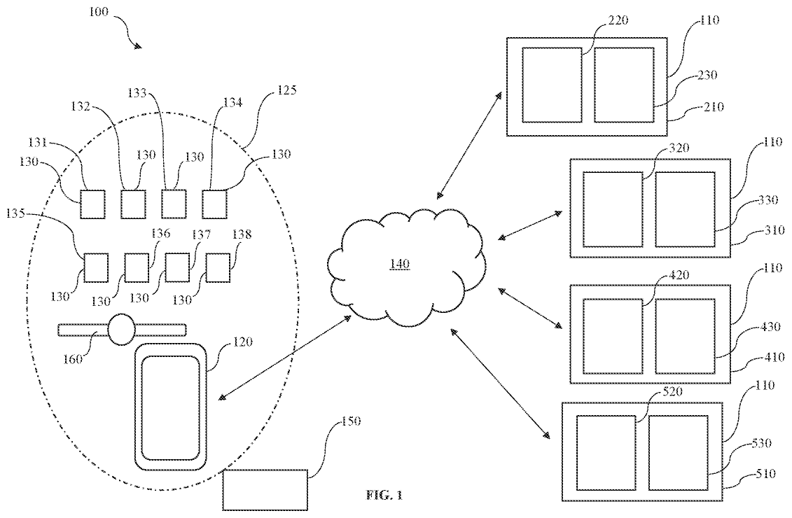

An example system for creating context-based personalized nutrition plans and recommendations (e.g., system 100) is shown, for example, in FIG. 1. The system 100 may include one or more servers 110, an electronic device 120, a plurality of sensors 130 in communication with the electronic device 120, and a network 140 over which the electronic device 120 may communicate with the one or more servers 110. In some embodiments, system 100 may include one or more wearable devices 160 (such as a watch) that are configured to communicate with electronic device 120 (e.g., via a personal area network 125) and/or with servers 110 over network 140. In some embodiments, system 100 may also include a genetic testing kit 150. Genetic testing kit 150 may be configured to determine genetic information of an individual.

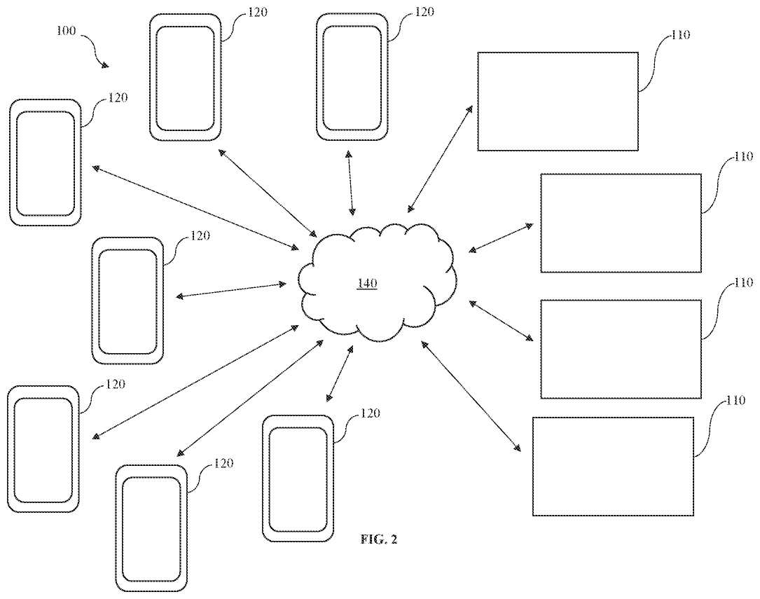

In some embodiments, network 140 comprises a wireless network, such as a wireless wide area network. For example, network 140 may be the internet. Network 140 may allow electronic device 120 to communicate with servers 110. In some embodiments, network 140 also allows communication between multiple electronic devices 120 (see FIG. 2).

In some embodiments, the one or more servers 110 comprise one or more databases storing, among other things, contextual information for multiple users (e.g., determinants of nutrition), information relating to nutrient sources/routes, and a mapping between micronutrients and determinants of nutrition. In some embodiments, system 100 only includes one server 110. In some embodiments, different servers 110 are used for different databases. An example is shown in FIG. 1. For example, one server 110 in system 100 may be server 210 that includes a processor 220 and a storage 230, which comprises a food micronutrient database. The food micronutrient database includes food nutrient data for a plurality of foods. The plurality of foods may include different foods and dishes from all over the world. In some embodiments, these foods and dishes include home-cooked dishes or recipes, food products sold at a grocery store, ingredients for a recipe, menu items from restaurants, or any other foods or dishes. Detailed information regarding each of these foods and dishes may be included in the food micronutrient database. For example, for each food, the food nutrient data may include proximates data, inorganics data, micronutrients data, vitamin fractions data, fatty acid compositions data, and/or bioactive compounds data. Thus, a complete picture of the nutrients obtained by consuming a particular food is stored in the food micronutrient database. In some embodiments, the food nutrient data may be obtained from companies that provide the various food items (e.g., grocery stores, restaurants, producers, manufacturers, etc.). As an alternative to storing this information, server 210 may be configured to access this data from other databases or otherwise pull this information from a resource accessible over the internet (network 140). In some embodiments, the food micronutrient database of server 210 may have any of the features of the food micronutrient databases disclosed in U.S. patent application Ser. No. 17/876,383, filed Jul. 28, 2022, which is incorporated herein by reference.

One server 110 in system 100 may be server 310 that includes a processor 320 and a storage 330, which comprises an oral supplement micronutrient database. Although multiple databases are referenced within this disclosure for storing various nutrients, micronutrients, therapies and other data, it will be understood that each of the various databases, though referenced as separate databases by name, may be part of one larger database and included within different sub-tables of the same database, or merely be part of one or multiple databases with distinguishing data elements to distinguish the differently named database classifications.

With that understanding, the oral supplements micronutrient database includes nutrient data for a plurality of oral supplements. A complete picture of the nutrients obtained by taking a particular oral supplement is stored in the oral supplement micronutrient database. In some embodiments, the nutrient data is provided by the manufacturer or the provider of each oral supplement. As an alternative to storing this information, server 310 may be configured to access this data from other databases or otherwise pull this information from a resource accessible over the internet (network 140).

The inhaled nutrition therapy micronutrient database includes nutrient data for a plurality of inhaled therapies. A complete picture of the nutrients obtained by taking a particular inhaled nutrition therapy is stored in the inhaled nutrition therapy micronutrient database. In some embodiments, the nutrient data is provided by the manufacturer or the provider of each inhaled nutrition therapy. As an alternative to storing this information, server 310 may be configured to access this data from other databases or otherwise pull this information from a resource accessible over the internet (network 140).

One server 110 in system 100 may be server 410 that includes a processor 420 and a storage 430, which comprises an IV micronutrient database. The IV micronutrient database includes nutrient data for a plurality of IV formulas. The IV formulas may be existing formulas which are selected based on an individual's micronutrient needs, as disclosed in U.S. patent application Ser. No. 17/876,352, filed Jul. 28, 2022, which is incorporated herein by reference. In some embodiments, processor 420 may be used to develop a new IV formula (e.g., by modifying an existing formula, creating a new formula from scratch, etc.) based on an individual's micronutrient needs, as disclosed in U.S. patent application Ser. No. 17/876,352, filed Jul. 28, 2022, or U.S. patent application Ser. No. 17/880,920, filed Aug. 4, 2022, which are incorporated herein by reference. A complete picture of the nutrients obtained by receiving a particular IV formula is stored in the IV micronutrient database. In some embodiments, the nutrient data is provided by the manufacturer or the provider of each IV formula. As an alternative to storing this information, server 410 may be configured to access this data from other databases or otherwise pull this information from a resource accessible over the internet (network 140).

One server 110 in system 100 may be server 510 that includes a processor 520 and a storage 530. Server 510, in some embodiments, may be a third-party server associated with a different app or service on an electronic device 120, such as a fitness tracking app or service. Storage 530 may comprise health and fitness information associated with the fitness tracking app or service. This is just one example. In other examples, server 510 may be associated with any other app or service that relates to any determinant of nutrition (additional determinants of nutrition are discussed below). Providing system 100 with access to server 510 may improve the functionality of system 100 and other systems and methods disclosed herein.

Other servers may also be included, similar to the ones discussed above. For example, there may be a server 110 dedicated to a topical nutrient database or an inhaled nutrient database. As another example, there may be a server 110 dedicated to an IM micronutrient database (similar to the IV micronutrient database). Alternatively, any of the databases discussed herein may be combined into a single server 110.

In some embodiments, there may be a server 110 dedicated to a user library, which stores a profile for each individual that uses system 100 (for example, each recipient of food, oral supplements, topical supplements, IV formula, IM formula, etc. based on using system 100). The user profile may include the genetic data of the user, the medical data of the user, the medical history of the user, the personal therapeutic objectives of the user, lifestyle information, habits, stresses, and other contextual information of the user. Thus, the profile may contain and track any or all of the determinants of nutrition discussed herein for that particular user. In some embodiments, users may enter or update this data via their electronic devices 120. In some embodiments, server 110 may retrieve this data from other apps or services associated with the user's electronic device and/or from server 510. In some embodiments, other users (e.g., healthcare providers) may gain access to another user's profile to enter or update data (e.g., genetic data, medical data, etc.).

In some embodiments, there may be a server 110 dedicated to a mapping of contextual information to micronutrients. The mapping may have any of the features or use any of the principles disclosed with respect to the mappings disclosed in U.S. patent application Ser. No. 17/876,352, filed Jul. 28, 2022, U.S. patent application Ser. No. 17/876,383, filed Jul. 28, 2022, or U.S. patent application Ser. No. 17/880,920, filed Aug. 4, 2022, the disclosures of which are incorporated herein by reference in their entireties. As system 100 is used and feedback is received after individuals are treated with an identified micronutrient and a recommended route of delivering the micronutrient, the mapping on server 110 is constantly updated. The treatment of multiple individuals with different contextual information thus improves the system 100 and the methods disclosed herein, making it more accurate and specific to an individual's situation.

In some embodiments, there may be a main server 110 that electronic device 120 primarily communicates with, and the main server may retrieve information from other servers 110. The main server 110 may be any of the servers 110 discussed above or a separate server 110.

The processor(s) of servers 110 (e.g., processors 220, 320, 420, 520) may simply be used to retrieve information from the database(s) or storage(s) of servers 110 (e.g., storages 230, 330, 430, 530). For example, a processor 220, 320, 420, 520 may receive a request for information from electronic device 220 (e.g., a request for food options, oral supplement options, and/or IV or IM nutrition therapy formula options that provide a particular micronutrient, or a request for a lifestyle/health-related recommendation, etc.), and processor 220, 320, 420, 520 may retrieve the requested information and cause it to be sent over network 140 to electronic device 120. In this situation, the electronic device 120 would be completing more of the processing to perform the methods disclosed herein. In other embodiments, processor(s) of servers 110 may be configured to perform more of the disclosed methods at the server itself. In this situation, the server 110 would be completing more of the processing to perform the methods disclosed herein and provide the output to an individual at electronic device 120. Any division of the method steps disclosed herein between electronic device 120 and server 110 is within the scope of this disclosure. A user of electronic device 120 would likely not notice a difference between electronic device 120 or server 110 performing more of the processing. Additional details about the methods performed by server 110 and/or electronic device 120 are discussed below.

Electronic device 120 may be a smartphone, tablet, desktop computer, laptop computer, smartwatch, or some other type of electronic device. In some embodiments, electronic device 120 is configured to run multiple applications. One application may be dedicated to system 100 and the methods disclosed herein (e.g., for creating nutrition plans and recommendations and other health-related or lifestyle recommendations). In some embodiments, electronic device 120 is configured to receive information about the determinants of nutrition for a user, such as genetic information of the individual, medical information of the individual, therapeutic objectives of the individual, and lifestyle information of the individual, as well as other determinants of nutrition (e.g., culture, habits, environment, etc.) discussed herein. Determinants of nutrition information may be received from a variety of sources. For example, determinants of nutrition information may be received from one or more sensors 130 (discussed below), from a wearable device 160, from other applications on the electronic device 120 (discussed below), from user input onto a user interface, from a server (e.g., previously saved information), from a genetic test, and/or from a medical provider.

As one example of receiving information, the dedicated application for creating nutrition plans and recommendations on electronic device 120, in some embodiments, may have access to information related to other applications on electronic device 120, such as a fitness tracking application, a nutrition tracking application, a sleep tracking application, a health-related application, or any other application that collects data relating to a user's culture, lifestyle, environment, habits, or other determinants of nutrition. In some embodiments, a user may link accounts from the other applications to the dedicated application for creating nutrition plans and recommendations. The information related to other applications may be information that is obtained while using the other applications, or it may be any information stored in connection with the other applications or collected for the other applications. In some embodiments, the dedicated application for creating nutrition plans and recommendations obtains the information related to other applications by retrieving information that is stored on the electronic device 120 itself (e.g., from one of the other applications). In some embodiments, the dedicated application obtains the information from a server 110 associated with the other applications (e.g., server 510).

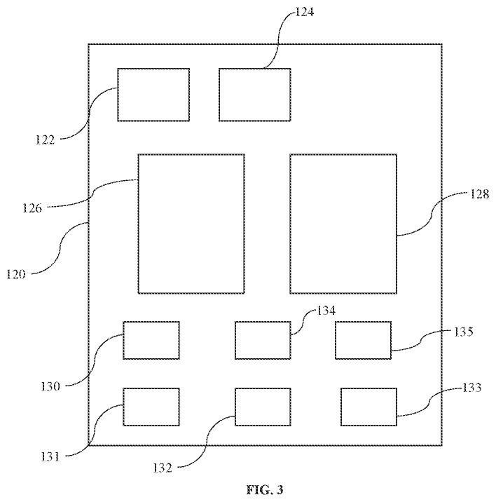

In some embodiments, system 100 includes a plurality of sensors 130 in communication with the electronic device 120. One or more of the sensors 130 may be part of electronic device 120. For example, there may be sensors 130 disposed within or on electronic device 120 (see FIG. 3). In some embodiments, one or more of the sensors 130 are separate from electronic device 120, for example, as a standalone device or as part of a wearable device 160. In some embodiments, system 100 may include at least one sensor 130 as part of electronic device 120 and at least one sensor 130 that is separate from electronic device 120. In some embodiments, sensors 130, wearable device(s) 160, and electronic device 120 may be in communication with each other as part of a personal area network 125. In some embodiments, sensors 130, wearable device(s) 160, and electronic device 120 are configured to communicate with each other via Bluetooth or some other wireless communication protocol. In some embodiments, sensors 130, wearable device(s) 160, and electronic device 120 are configured to communicate via a hard-wired connection. In some embodiments, genetic testing kit 150 may also be configured to communicate with electronic device 120 (e.g., via Bluetooth, as part of personal area network 125, via a hard-wired connection, etc.).

In some embodiments, system 100 includes different types of sensors 130. For example, sensors 130 may include a position sensor 131 (such as a GPS sensor), an accelerometer 132, a gyroscope 133, a pedometer 134, a heart rate sensor 135, a barometer 136, a thermometer 137, an air humidity sensor 138, or any other type of sensor 130 (e.g., such as a blood glucose monitor). For example, system 100 may include any sensor 130 that senses data related to determinants of nutrition. The data may be directly related to determinants of nutrition. For example, a heart rate sensor 135 may sense data (e.g., heart rate) that provides medical information of the user.

The data sensed by sensors 130 may also be indirectly related to determinants of nutrition. As one example, a position sensor 131 (such as a GPS sensor) may sense location data (and thus determine a location of the individual) that is then used to obtain information about determinants of nutrition. For example, air quality data associated with the location of the individual may be retrieved based on the location data sensed by the position sensor 131. Other data associated with the location of the individual may also be retrieved, such as weather data. Location data from position sensor 131 may be used for other purposes. For example, system 100 may make assumptions on cultural and environment determinants of nutrition based on the location data (e.g., assuming higher stress levels if the individual is located in a high-stress location such as work, assuming dietary restrictions depending on geographic location, etc.). As another example, system 100 may use location data from position sensor 131 to adjust nutrition plans and recommendations. If an individual is in a particular country or region that does not have access to a certain food, nutrition supplement, or IV therapy, etc. (or if the particular country does not allow it), system 100 may automatically adjust its recommendations to the best option that is available based on the user's location, even if that is not the best overall option. In some embodiments, system 100 may have region rules that are configured to adapt to a particular country or region.

In some embodiments, sensors 130 may be associated with another application on the electronic device (e.g., a fitness, nutrition, or health-related application). Thus, system 100 may retrieve data obtained by sensors from or for other applications. As noted above, this data may be obtained from other servers 510. Alternatively, data may be retrieved from an online database. In some embodiments, sensors 130 may be intended for use specifically with the systems and methods disclosed herein.

In some embodiments, system 100 may include multiple electronic devices 120, as shown, for example, in FIG. 2. Although FIG. 2 only shows servers 110, network 140, and electronic devices 120, each electronic device 120 in FIG. 2 may be associated with an individual that is using system 100. As such there may be a plurality of sensors 130, wearable device(s) 160, and/or a genetic testing kit 150 (as shown in FIG. 1) for each of these individuals. System 100 may include any number of electronic devices 120 (e.g., dozens, hundreds, thousands, millions, etc.). The more individuals that use system 100, the more accurate, specific, and complete the mapping will be, thus improving overall health of a society and reducing risk of chronic disease.

A schematic of an example electronic device 120 is shown in FIG. 3. In this example, electronic device 120 includes a position sensor 131, an accelerometer 132, a gyroscope 133, a pedometer 134, and a heart rate sensor 135. In some embodiments, electronic device 120 may have fewer sensors 130 or may have one or more additional sensors 130, as represented in FIG. 3. In some embodiments, electronic device 120 comprises a processor 122, a transceiver 124, a user interface 126, and/or a display 128. In some embodiments, processor 122 is configured to perform one or more of the operations of the methods disclosed herein (e.g., retrieve information, identify micronutrients, identify routes of nutrition that provide the micronutrients, make recommendations, etc.). In some embodiments, transceiver 124 is configured to send and receive information to other components of system 100, including servers 110 (e.g., servers 210, 310, 410, 510), other electronic devices 120, stand-alone sensors 130, and wearable devices 160. In some embodiments, electronic device 120 comprises multiple transceivers to facilitate different types of communication (e.g., over network 140, via personal area network 125, via Bluetooth, etc.). In some embodiments, user interface 126 is configured to receive input from an individual. The received input may be entry of information relating to determinants of nutrition, a request for a plan and/or a recommendation, feedback on previously consumed foods, supplements, or treatments, and/or user preferences. In some embodiments, display 128 is configured to display plans/recommendations, requests for the user (e.g., to enter information, to enter feedback, etc.), a user's profile/history, communications from a medical provider, etc. Although FIG. 3 schematically shows user interface 126 and display 128 separately, they may be combined (e.g., as a touch screen on an electronic device 120).

In some embodiments, system 100 contains one or more mappings of determinants of nutrition to micronutrients. The mappings may be based on scientific literature and/or results from previous food intake, supplement intake, and therapy treatments. The mappings may have the characteristics of the mappings disclosed in U.S. patent application Ser. No. 17/876,352, filed Jul. 28, 2022, U.S. patent application Ser. No. 17/876,383, filed Jul. 28, 2022, or U.S. patent application Ser. No. 17/880,920, filed Aug. 4, 2022, the disclosures of which are incorporated herein by reference in their entireties. Every individual has different determinants of nutrition through, for example, leading different lifestyles, facing different exposures, following particular behaviors, and/or having different genotypes. In order to make appropriate plans/recommendations for a particular individual, system 100 collects determinants of nutrition information for that particular individual. The determinants of nutrition may include medical information, therapeutic objectives, lifestyle information, habits, stress levels, and genetic information.

Examples of medical information include the individual's personal medical history, family medical history, diseases of which the patient is at heightened risk, medical and surgical procedures, microbiome, current symptoms, medical diagnoses, blood test results, vital signs (e.g., blood pressure, heart rate, heart rhythm, etc.), and allergies. Medical information may include biodata that is captured and measured through various processes, which may include blood testing, urine sampling (e.g., toxicology screens, etc.), stool sampling, continuous blood glucose monitoring, and imaging/scanning (e.g., ultrasound, etc.). Devices to collect medical information may include blood pressure monitors, heart rate monitors, body hydration monitors, breathalyzers, blood glucose monitors, metabolic devices, pulse oximeters, and wearable devices configured to measure any type of biodata. Other medical information may also be used as determinants of nutrition.

Examples of therapeutic objectives include health goals, aesthetic goals, or fitness goals. The therapeutic objectives may be to lose weight, to gain weight, to improve fitness level, to improve energy levels, to alter skin tone (to either a darker tone or a lighter tone), to improve skin quality and/or health, to improve hair quality and/or health, to improve mental acuity, to prevent disease, to improve symptoms of an existing illness or disease, to improve sleep quality, to improve longevity/anti-aging, to protect against excessive stress, to protect against toxin exposures (e.g., from pollution, chemicals, alcohol, etc.), to rehydrate, to support immune health, and/or to heal a wound. Other therapeutic objectives may also be used.

Examples of lifestyle information include the individual's physical activities, mental activities, occupation, social network, current and/or prior food choices and diets, financial security, culture/customs, preferences, geographical location, travel, and exposure to pathogens, chemicals, pollution, heavy metals, and radiation. Some of these determinants of nutrition may be related to each other. In addition, some of these determinants of nutrition may be used to obtain additional determinants of nutrition. For example, geographical location may be used to obtain information about pollution levels/air quality, weather, and other geographical influences that may affect the nutrient demand of an individual. Whether an individual lives in a polluted area of the world (and thus has poor detoxification) may affect the nutrient demands of that individual and should be considered. As another example, geographical location may be used to determine an individual's social network (e.g., by determining who the individual spends time with). Thus, the use of system 100 goes far beyond replicating a doctor's recommendations because it utilizes far more data and understands how the data influences nutrient demands of an individual and what an outcome will be for a given set of circumstances. Other lifestyle information may also be used as determinants of nutrition.

Habits and stress levels may be considered a sub-category of lifestyle information. Examples of habits that are of interest as determinants of nutrition include smoking, excessive alcohol consumption, recreational drug use, and participation in dangerous activities. Examples of stress that are of interest as determinants of nutrition include physical stress and mental stress. Other habits and stress levels may also be used as determinants of nutrition.

Examples of genetic information include the individual's gene, genotype, genome, single-nucleotide polymorphism (SNP), genetic profile, epigenetic profile, or other piece of genetic data. In some embodiments, the genetic information comprises epigenetic measurements (e.g., epigenetic clocks, methylation scores, etc.). Other genetic information may also be used as determinants of nutrition.

In some embodiments, determinants of nutrition may be received by user input at electronic device 120. In some embodiments, determinants of nutrition may be received from other applications on electronic device 120. In some embodiments, determinants of nutrition may be received via sensors 130 and/or wearable 160. In some embodiments, determinants of nutrition may be retrieved based on data received by sensors 130. In some embodiments, determinants of nutrition may be determined based on data received by sensors 130 and/or information input by a user into electronic device 120. System 100 may be configured to use whatever determinants of nutrition for a particular individual that are available to identify micronutrients for a nutrition plan/recommendation and to recommend a route of providing nutrition. Similarly, system 100 may be configured to use whatever determinants of nutrition for a particular individual that are available to evaluate feedback of how a particular micronutrient/route of nutrition worked to inform and update the mapping for future users.

Once one or more micronutrients have been identified (based on an individual's determinants of nutrition and the mapping stored at server 110), system 100 may be configured to recommend a particular route or delivery method of the micronutrient(s). The micronutrient(s) may be delivered via food, oral supplements, intravenous nutrition therapy formulas, intramuscular nutrition therapy formulas, or topical supplements. As discussed above, server 110 may contain one or more databases for these routes of nutrition that provide a complete picture of the nutrients received by consuming/taking/receiving the food, oral supplements, IV formula, IM formula, and topical supplements. Other delivery routes may also be used. System 100 may recommend the route of nutrition depending on effectiveness for the individual's unique context, cost, user preference, availability, and other factors.

Thus, using the information received/retrieved/determined relating to the determinants of nutrition, system 100 provides a precision nutrition suite of products and services that takes each user's individuality and merges it with different routes to nutrition (e.g., food, oral supplements, IV, etc.) and is able to monitor efficacy and safety on a global scale. With the determinants of nutrition information, system 100 can use artificial intelligence and machine learning to observe for patterns and trends that can revolutionize scientific discovery, relevant to nutritional health, and can develop new products and services to enhance health and wellness. Thus, system 100 may help prevent disease, slow signs of aging, maintain metabolic function, and foster growth and development.

In some embodiments, system 100 is configured to automatically map a symptom/disease/disorder of an individual person, the individual's genetics, the individual's personal situation, and the individual's health goals (i.e., determinants of nutrition) to one or more micronutrients known to be clinically effective for treating that patient's specific situation, and to recommend a nutrient therapy (IV or IM), supplement (oral or topical), or food that will help the individual. The system 100 is also configured to subsequently obtain feedback on how the recommended route of nutrition worked and then update a mapping of determinants of nutrition to micronutrients (and/or routes of nutrition) to treat the next person. Thus, the effectiveness of system 100 improves over time as a more complete mapping of how each determinant of nutrition and combination of determinants affects the micronutrients that are needed and the effectiveness of each route of nutrition.

In some embodiments, system 100 is used to perform methods of creating a context-based personalized nutrition recommendation for reducing risk of disease. At a high level, as the discussion above shows, the method may include receiving information about an individual (e.g., determinants of nutrition), identifying one or more micronutrients based on the individual's determinants of nutrition, identifying one or more routes of nutrition that provide the identified one or more micronutrients, and recommending one of the routes of nutrition based on a number of factors.

In some embodiments, receiving information about an individual comprises receiving any determinants of nutrition discussed in this application and any combination of these determinants of nutrition. As one example, receiving information about the individual comprises receiving genetic information, medical information, and therapeutic objectives of the individual. In some embodiments, receiving information about an individual also comprises receiving lifestyle information about the individual. In some embodiments, the lifestyle information comprises information about physical activities, mental activities, occupation, social network, financial security, culture, habits, and preferences of the individual. This information may be received at electronic device 120 (e.g., through user input at user interface 126, from sensors 130, from wearable device 160, or from servers 110). In some embodiments, the genetic information is determined by a genetic test (e.g., genetic testing kit 150). This information may be sent over network 140 and/or personal area network 125 to electronic device 120, or it may be entered by a user through user interface 126.

In some embodiments, a location of the individual is determined using position sensor 131. In some embodiments, data associated with the location of the individual is retrieved. The data may include real-time air quality data. Thus, electronic device 120 may be configured to determine real-time air quality data based on location data form the position sensor 131. Other data associated with the location of the individual that may be retrieved includes weather, cultural and environmental data, and available options of routes of nutrition at the location of the individual (e.g., available options of food, oral supplements, topical treatments, inhaled treatments, intravenous nutrition therapy formulas, and intramuscular nutrition therapy formulas).

In some embodiments, fitness data of the individual is collected using wearable device 160. For example, an accelerometer 132 (which may be part of wearable device 160) may collect motion data and a heart rate sensor 135 or monitor (which may be part of wearable device 160) may collect heart rate data. In some embodiments, electronic device 120 is configured to determine fitness data of the individual based on the motion data and the heart rate data.

In some embodiments, identifying one or more micronutrients for the individual based on the individual's determinants of nutrition may comprise identifying one or more micronutrients based on the genetic information, medical information, therapeutic objectives, lifestyle information, and fitness data of the individual and based on the real-time air quality data associated with the location of the individual. In some embodiments, system 100 is configured to identify the micronutrients in response to a recommendation request from an individual. For example, a recommendation request may be received at electronic device 120 by the individual using electronic device 120. In response to the recommendation request, electronic device 120 may identify one or more micronutrients. For example, electronic device 120 may communicate with server(s) 110 over network 140 to retrieve relevant details from the mapping of determinants of nutrition to micronutrients and thereby identify the micronutrients that are suitable for the individual given the contextual information (i.e., the determinants of nutrition) for that particular individual.

After identifying the micronutrient(s), routes of nutrition that provide the micronutrient(s) may be identified. In some embodiments, identifying one or more routes of nutrition that provide the identified one or more micronutrients comprises identifying at least two of a food, an oral supplement, a topical treatment, an inhaled nutrition treatment, an intravenous nutrition therapy formula, and an intramuscular nutrition therapy formula that provide the identified one or more micronutrients. For example, a food and an oral supplement may be identified, both of which contain the identified micronutrient(s). In some embodiments, a food, an oral supplement, a topical treatment, an inhaled nutrition treatment, an intravenous nutrition therapy formula, and an intramuscular nutrition therapy formula may all be identified. In some embodiments, multiple options under each route of nutrition may be identified (e.g., two or more foods, two or more oral supplements, etc.). Electronic device 120 may identify the routes of nutrition by communicating with server(s) 110 over network 140 to retrieve the options of routes of nutrition from the databases discussed above that provide the identified micronutrient(s).

After the routes of nutrition have been identified, system 100 may recommend one of the identified routes based on a number of factors. In some embodiments, electronic device 120 makes the recommendation. For example, electronic device 120 may recommend one of the food, the oral supplement, the topical treatment, the inhaled nutrition treatment, the intravenous nutrition therapy formula, and the intramuscular nutrition therapy formula based on a cost of each of the food, the oral supplement, the topical treatment, the inhaled nutrition treatment, the intravenous nutrition therapy formula, and the intramuscular nutrition therapy formula, the financial security of the individual, and an effectiveness of each of the food, the oral supplement, the topical treatment, the inhaled nutrition treatment, the intravenous nutrition therapy formula, and the intramuscular nutrition therapy formula in providing the identified one or more micronutrients. Other factors may also be considered, such as the availability of the particular route of nutrition at the location of the individual, the ease in securing the route of nutrition, the individual's preferences, etc.

Electronic device 120 may be configured to balance the factors that affect the recommendation in different ways. In some embodiments, a user can change how the electronic device 120 is configured to balance the factors, which may be based on the user's desires/preferences, suggestions from the user's medical provider, and/or certain situations. In some embodiments, electronic device 120 is configured to recommend the most effective treatment in providing the identified micronutrient(s) regardless of cost. In some embodiments, electronic device 120 is configured to recommend the most cost-effective route of nutrition that provides at least some benefit for the user's particular situation by providing the identified micronutrient(s) (even if less effective than other routes). In some embodiments, electronic device 120 is configured to optimize effectiveness and cost. For example, if one nutrient source is expensive but only slightly better than another much more affordable approach (such as taking vitamin c pills rather than eating oranges), electronic device 120 may recommend the less expensive, nearly as effective option.

Whether electronic device 120 prioritizes cost or effectiveness may be a setting that a user selects, or electronic device 120 may be configured to base this priority on other factors, such as the financial security of the individual. For example, where an individual has low financial security, electronic device 120 may prioritize cost and recommend the less expensive option. Thus, system 100 may provide an inexpensive food recommendation to help a user's condition or symptom as an alternative to immediately going to a doctor. On the other hand, where an individual has high financial security, electronic device 120 may prioritize effectiveness and give less weight to cost. In some embodiments, electronic device 120 may give multiple options, each indicating the effectiveness and the cost (or other characteristics), while allowing the user to select which option to take.

In some embodiments, the determinants of nutrition affect the final recommendation of which route of nutrition. For example, an individual that has diabetes may be given a recommendation (e.g., by electronic device 120) that is an oral supplement rather than a food that contains too much sugar.

In some embodiments, the recommending is performed by a first application on electronic device 120 (such as the dedicated application discussed above). In some embodiments, receiving lifestyle information comprises retrieving data associated with a second application on the electronic device 120 (such as the other applications discussed above).

In some embodiments, the method comprises developing a nutrition plan based on the determinants of nutrition (e.g., the genetic information, medical information, therapeutic objectives, lifestyle information, and fitness data of the individual). The nutrition plan may be adjusted based on the real-time air quality data associated with the location of the individual. The nutrition plan may also be adjusted based on updated medical information, therapeutic objectives, or lifestyle information of the individual.

In some embodiments, system 100 may be used in specific contexts. For example, in some embodiments, system 100 may be used while shopping. In some embodiments, electronic device 120 is configured to scan food items at a store (e.g., a grocery store) using a dedicated scanner or a camera on the electronic device 120. Electronic device 120 may then determine which food item is best for the individual based on the individual's genetic information and other determinants of nutrition. A similar approach may be used for online shopping with the electronic device 120 either scanning an identifier of the item or, when the user is using another application on the electronic device 120 to shop, communicating with the other application that the individual is using to shop online. A similar approach may also be used at a restaurant when deciding on a menu item. In some embodiments, system 100 may provide recommendations of particular foods or nutrients to avoid (e.g., avoid salty foods today, etc.)

In some embodiments, system 100 may be used while purchasing oral supplements, topical supplements, or medicines at a pharmacy. For example, electronic device 120 may scan a supplement, access the individual's medical records and purchase history, and/or provide recommendations based on the individual's genetic information and other determinants of nutrition. In addition, the methods disclosed herein may alternatively be used for beauty products applied to an individual's skin to identify the products that are most suitable for the individual's genetic, medical, and other contextual information.

In some embodiments, system 100 may provide recommendations other than nutrient recommendations. For example, system 100 may provide a recommendation to alter one's lifestyle, such as increasing physical activity, eliminating bad habits, changing environments, moving to a new geographic location, or changing jobs, etc. This may be appropriate in instances where a suitable micronutrient source is hard to identify given the particular determinants of nutrition or where the micronutrients alone are insufficient to resolve a particular symptom or condition.

In some embodiments, system 100 may be used to create and/or modify food items, oral supplements, topical supplements, IV nutrition therapy formulas, or IM nutrition therapy formulas. For example, as the mapping continues to be updated, manufacturers or producers of these routes of nutrition may identify certain populations or categories of people that would benefit from a different or modified combination of micronutrients and design a supplement, food, or therapy formula for that group of people. Alternatively, the manufacturers or producers may learn that a certain supplement, food, or therapy formula is not effective and may make adjustments to improve its effectiveness.

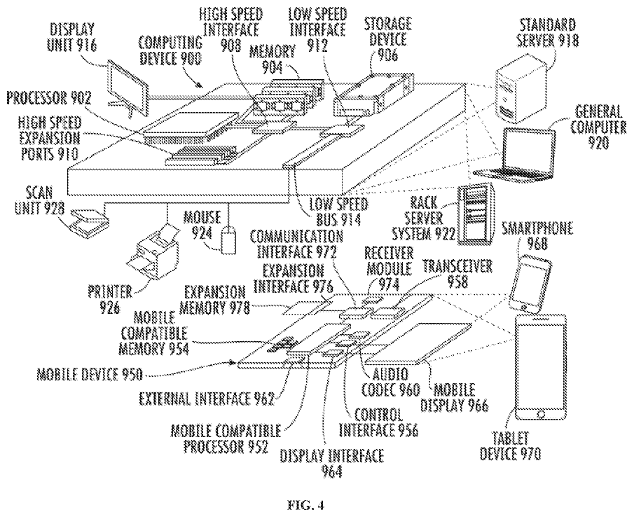

As discussed above, the systems and methods disclosed herein may be partially or fully implemented with a computer through software running on or associated with the computer, or an application accessible by the computer or some other electronic device. As one example, FIG. 4 is a schematic diagram of specific computing device 900 and a specific mobile computing device 950 that can be used to perform and/or implement any of the embodiments disclosed herein.

The specific computing device 900 may represent various forms of digital computers, such as laptops, desktops, workstations, personal digital assistants, servers, blade servers, mainframes, and/or other appropriate computers. The specific mobile computing device 950 may represent various forms of mobile devices, such as smartphones, camera phones, personal digital assistants, cellular telephones, and other similar mobile devices. The components shown here, their connections, couples, and relationships, and their functions, are meant to be exemplary only, and are not meant to limit the embodiments described and/or claimed, according to one embodiment.

The specific computing device 900 may include a processor 902, a memory 904, a storage device 906, a high-speed interface 908 coupled to the memory 904 and a plurality of high-speed expansion ports 910, and a low-speed interface 912 coupled to a low-speed bus 914 and a storage device 906. In one embodiment, each of the components heretofore may be inter-coupled using various buses, and may be mounted on a common motherboard and/or in other manners as appropriate. The processor 902 may process instructions for execution in the specific computing device 900, including instructions stored in the memory 904 and/or on the storage device 906 to display a graphical information for a GUI on an external input/output device, such as a display unit 916 coupled to the high-speed interface 908, according to one embodiment.

In other embodiments, multiple processors and/or multiple buses may be used, as appropriate, along with multiple memories and/or types of memory. Also, a plurality of specific computing devices 900 may be coupled with, with each device providing portions of the necessary operations (e.g., as a server bank, a group of blade servers, and/or a multi-processor system).

The memory 904 may be coupled to the specific computing device 900. In one embodiment, the memory 904 may be a volatile memory. In another embodiment, the memory 904 may be a non-volatile memory. The memory 904 may also be another form of computer-readable medium, such as a magnetic and/or an optical disk. The storage device 906 may be capable of providing mass storage for the specific computing device 900. In one embodiment, the storage device 906 may be a floppy disk device, a hard disk device, an optical disk device, a tape device, a flash memory and/or other similar solid state memory device. In another embodiment, the storage device 906 may be an array of the devices in a computer-readable medium previously mentioned heretofore, including devices in a storage area network and/or other configurations.

A computer program may be comprised of instructions that, when executed, perform one or more methods, such as those described above. The instructions may be stored in the memory 904, the storage device 906, a memory coupled to the processor 902, and/or a propagated signal.

The high-speed interface 908 may manage bandwidth-intensive operations for the specific computing device 900, while the low-speed interface 912 may manage lower bandwidth-intensive operations. Such allocation of functions is exemplary only. In one embodiment, the high-speed interface 908 may be coupled to the memory 904, the display unit 916 (e.g., through a graphics processor and/or an accelerator), and to the plurality of high-speed expansion ports 910, which may accept various expansion cards.

In the embodiment, the low-speed interface 912 may be coupled to the storage device 906 and the low-speed bus 914. The low-speed bus 914 may be comprised of a wired and/or wireless communication port (e.g., a Universal Serial Bus (“USB”), a Bluetooth® port, an Ethernet port, and/or a wireless Ethernet port). The low-speed bus 914 may also be coupled to the scan unit 928, a printer 926, a keyboard, a mouse 924, and a networking device (e.g., a switch and/or a router) through a network adapter.

The specific computing device 900 may be implemented in a number of different forms, as shown in the figure. In one embodiment, the specific computing device 900 may be implemented as a standard server 918 and/or a group of such servers. In another embodiment, the specific computing device 900 may be implemented as part of a rack server system 922. In yet another embodiment, the specific computing device 900 may be implemented as a general computer 920 such as a laptop or desktop computer. Alternatively, a component from the specific computing device 900 may be combined with another component in a specific mobile computing device 950. In one or more embodiments, an entire system may be made up of a plurality of specific computing device 900 and/or a plurality of specific computing device 900 coupled to a plurality of specific mobile computing device 950.

In one embodiment, the specific mobile computing device 950 may include a mobile compatible processor 952, a mobile compatible memory 954, and an input/output device such as a mobile display 966, a communication interface 972, and a transceiver 958, among other components. The specific mobile computing device 950 may also be provided with a storage device, such as a microdrive or other device, to provide additional storage. In one embodiment, the components indicated heretofore are inter-coupled using various buses, and several of the components may be mounted on a common motherboard.

The mobile compatible processor 952 may execute instructions in the specific mobile computing device 950, including instructions stored in the mobile compatible memory 954. The mobile compatible processor 952 may be implemented as a chipset of chips that include separate and multiple analog and digital processors. The mobile compatible processor 952 may provide, for example, for coordination of the other components of the specific mobile computing device 950, such as control of user interfaces, applications run by the specific mobile computing device 950, and wireless communication by the specific mobile computing device 950.

The mobile compatible processor 952 may communicate with a user through the control interface 956 and the display interface 964 coupled to a mobile display 966. In one embodiment, the mobile display 966 may be a Thin-Film-Transistor Liquid Crystal Display (“TFT LCD”), an Organic Light Emitting Diode (“OLED”) display, and another appropriate display technology. The display interface 964 may comprise appropriate circuitry for driving the mobile display 966 to present graphical and other information to a user. The control interface 956 may receive commands from a user and convert them for submission to the mobile compatible processor 952.

In addition, an external interface 962 may be in communication with the mobile compatible processor 952, so as to enable near area communication of the specific mobile computing device 950 with other devices. External interface 962 may provide, for example, for wired communication in some embodiments, or for wireless communication in other embodiments, and multiple interfaces may also be used.

The mobile compatible memory 954 may be coupled to the specific mobile computing device 950. The mobile compatible memory 954 may be implemented as a volatile memory and a non-volatile memory. The expansion memory 978 may also be coupled to the specific mobile computing device 950 through the expansion interface 976, which may comprise, for example, a Single In Line Memory Module (“SIMM”) card interface. The expansion memory 978 may provide extra storage space for the specific mobile computing device 950, or may also store an application or other information for the specific mobile computing device 950.

Specifically, the expansion memory 978 may comprise instructions to carry out the processes described above. The expansion memory 978 may also comprise secure information. For example, the expansion memory 978 may be provided as a security module for the specific mobile computing device 950, and may be programmed with instructions that permit secure use of the specific mobile computing device 950. In addition, a secure application may be provided on the SIMM card, along with additional information, such as placing identifying information on the SIMM card in a non-hackable manner.

The mobile compatible memory may include a volatile memory (e.g., a flash memory) and a non-volatile memory (e.g., a non-volatile random-access memory (“NVRAM”)). In one embodiment, a computer program comprises a set of instructions that, when executed, perform one or more methods. The set of instructions may be stored on the mobile compatible memory 954, the expansion memory 978, a memory coupled to the mobile compatible processor 952, and a propagated signal that may be received, for example, over the transceiver 958 and/or the external interface 962.

The specific mobile computing device 950 may communicate wirelessly through the communication interface 972, which may be comprised of a digital signal processing circuitry. The communication interface 972 may provide for communications using various modes and/or protocols, such as a Global System for Mobile Communications (“GSM”) protocol, a Short Message Service (“SMS”) protocol, an Enhanced Messaging System (“EMS”) protocol, a Multimedia Messaging Service (“MIMS”) protocol, a Code Division Multiple Access (“CDMA”) protocol, Time Division Multiple Access (“TDMA”) protocol, a Personal Digital Cellular (“PDC”) protocol, a Wideband Code Division Multiple Access (“WCDMA”) protocol, a CDMA2000 protocol, and a General Packet Radio Service (“GPRS”) protocol.

Such communication may occur, for example, through the transceiver 958 (e.g., radio-frequency transceiver). In addition, short-range communication may occur, such as using a Bluetooth®, Wi-Fi, and/or other such transceiver. In addition, a GPS (“Global Positioning System”) receiver module 974 may provide additional navigation-related and location-related wireless data to the specific mobile computing device 950, which may be used as appropriate by a software application running on the specific mobile computing device 950.

The specific mobile computing device 950 may also communicate audibly using an audio codec 960, which may receive spoken information from a user and convert it to usable digital information. The audio codec 960 may likewise generate audible sound for a user, such as through a speaker (e.g., in a handset smartphone of the specific mobile computing device 950). Such a sound may comprise a sound from a voice telephone call, a recorded sound (e.g., a voice message, a music files, etc.) and may also include a sound generated by an application operating on the specific mobile computing device 950.

The specific mobile computing device 950 may be implemented in a number of different forms, as shown in the figure. In one embodiment, the specific mobile computing device 950 may be implemented as a smartphone 968. In another embodiment, the specific mobile computing device 950 may be implemented as a personal digital assistant (“PDA”). In yet another embodiment, the specific mobile computing device, 950 may be implemented as a tablet device 970.

Various embodiments of the systems and techniques described here can be realized in a digital electronic circuitry, an integrated circuitry, a specially designed application specific integrated circuits (“ASICs”), a piece of computer hardware, a firmware, a software application, and a combination thereof. These various embodiments can include embodiment in one or more computer programs that are executable and/or interpretable on a programmable system including one programmable processor, which may be special or general purpose, coupled to receive data and instructions from, and to transmit data and instructions to, a storage system, one input device, and at least one output device.

These computer programs (also known as programs, software, software applications, and/or code) comprise machine-readable instructions for a programmable processor, and can be implemented in a high-level procedural and/or object-oriented programming language, and/or in assembly/machine language. As used herein, the terms “machine-readable medium” and/or “computer-readable medium” refers to any computer program product, apparatus and/or device (e.g., magnetic discs, optical disks, memory, and/or Programmable Logic Devices (“PLDs”)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here may be implemented on a computing device having a display device (e.g., a cathode ray tube (“CRT”) and/or liquid crystal (“LCD”) monitor) for displaying information to the user and a keyboard and a mouse by which the user can provide input to the computer. Other kinds of devices can be used to provide for interaction with a user as well; for example, feedback provided to the user can be any form of sensory feedback (e.g., visual feedback, auditory feedback, and/or tactile feedback) and input from the user can be received in any form, including acoustic, speech, and/or tactile input.

The systems and techniques described here may be implemented in a computing system that includes a back end component (e.g., as a data server), a middleware component (e.g., an application server), a front end component (e.g., a client computer having a graphical user interface, and/or a Web browser through which a user can interact with an embodiment of the systems and techniques described here), and a combination thereof. The components of the system may also be coupled through a communication network.

The communication network may include a local area network (“LAN”) and a wide area network (“WAN”) (e.g., the Internet). The computing system can include a client and a server. In one embodiment, the client and the server are remote from each other and interact through the communication network.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It may be appreciated that the various systems, methods, and apparatus disclosed herein may be embodied in a machine-readable medium and/or a machine accessible medium compatible with a data processing system (e.g., a computer system), and/or may be performed in any order.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

Where the above examples, embodiments and implementations reference examples, it should be understood by those of ordinary skill in the art that other use case, execution environments, and data structures could be intermixed or substituted with those provided. In places where the description above refers to particular embodiments of a personalized therapy system or method, it should be readily apparent that a number of modifications may be made without departing from the spirit thereof and that these embodiments and implementations may be applied to other personalized therapy systems and methods as well. Accordingly, the disclosed subject matter is intended to embrace all such alterations, modifications and variations that fall within the spirit and scope of the disclosure and the knowledge of one of ordinary skill in the art.

The concepts disclosed herein are not limited to the specific examples shown herein. It should be readily apparent that a number of modifications may be made without departing from the spirit thereof and that these implementations may be applied to other implementations disclosed or undisclosed. The presently disclosed methods and systems are, therefore, to be considered in all respects as illustrative and not restrictive.

What is claimed is:

1. A method of creating a context-based personalized nutrition recommendation, the method comprising:
receiving genetic information, medical information, and therapeutic objectives of an individual, the genetic information determined by a genetic test;
receiving lifestyle information about the individual, wherein the lifestyle information comprises information about physical activities, mental activities, occupation, social network, financial security, culture, habits, and preferences of the individual;
using a position sensor, determining a location of the individual;
retrieving data associated with the location of the individual from the position sensor, wherein the data comprises real-time air quality data;
collecting fitness data of the individual using a wearable device;
identifying one or more micronutrients for the individual based on the genetic information, medical information, therapeutic objectives, lifestyle information, and fitness data of the individual and based on the real-time air quality data associated with the location of the individual;
identifying at least two of a food, an oral supplement, a topical treatment, inhaled nutrition treatment, an intravenous nutrition therapy formula, and an intramuscular nutrition therapy formula that provide the identified one or more micronutrients; and
recommending one of the identified at least two of the food, the oral supplement, the topical treatment, the inhaled nutrition treatment, the intravenous nutrition therapy formula, and the intramuscular nutrition therapy formula based on a cost of each of the identified at least two of the food, the oral supplement, the topical treatment, the inhaled nutrition treatment, the intravenous nutrition therapy formula, and the intramuscular nutrition therapy formula, the financial security of the individual, and an effectiveness of each of the at least two of the food, the oral supplement, the topical treatment, the inhaled nutrition treatment, the intravenous nutrition therapy formula, and the intramuscular nutrition therapy formula in providing the identified one or more micronutrients.

2. The method of claim 1, wherein the recommending is performed by a first application on an electronic device, and wherein receiving lifestyle information comprises retrieving data associated with a second application on the electronic device.

3. The method of claim 1, wherein the data associated with the location of the individual comprises available options of food, oral supplements, topical treatments, inhaled nutrition treatments, intravenous nutrition therapy formulas, and intramuscular nutrition therapy formulas at the location of the individual.

4. The method of claim 1, further comprising receiving a recommendation request from the individual.

5. The method of claim 1, further comprising developing a nutrition plan based on the genetic information, medical information, therapeutic objectives, lifestyle information, and fitness data of the individual.

6. The method of claim 5, further comprising adjusting the nutrition plan based on the real-time air quality data associated with the location of the individual.

7. The method of claim 5, further comprising adjusting the nutrition plan based on updated medical information, therapeutic objectives, or lifestyle information of the individual.

8. A system for creating a context-based personalized nutrition recommendation, the system comprising:
a genetic test configured to determine genetic information of an individual;
an electronic device configured to receive the genetic information of the individual, medical information of the individual, therapeutic objectives of the individual, and lifestyle information of the individual;
a plurality of sensors in communication with the electronic device, the plurality of sensors comprising a position sensor, an accelerometer, and a heart rate monitor, wherein the electronic device is configured to determine real-time air quality data based on location data from the position sensor, and wherein the electronic device is configured to determine fitness data of the individual based on motion data from the accelerometer and heart rate data from the heart rate monitor; and
a server comprising a food database, an oral supplement database, a topical treatment database, an inhaled nutrition therapy database, an intravenous nutrition therapy database, an intramuscular nutrition therapy database, and a mapping of genetic information, medical information, therapeutic objectives, lifestyle information, air quality data, and fitness data to one or more micronutrients, wherein the server is in communication with the electronic device,
wherein the system is configured to:
use the mapping to identify one or more micronutrients for the individual based on the genetic information, medical information, therapeutic objectives, lifestyle information, and fitness data of the individual and based on the real-time air quality data;
identify at least two of a food from the food database, an oral supplement from the oral supplement database, a topical treatment from the topical treatment database, an inhaled nutrition treatment from an inhaled nutrition treatment database, an intravenous nutrition therapy formula from the intravenous nutrition therapy database, and an intramuscular nutrition therapy formula from the intramuscular nutrition therapy database that provide the identified one or more micronutrients; and
recommend one of the at least two of the food, the oral supplement, the topical treatment, the inhaled nutrition treatment, the intravenous nutrition therapy formula, and the intramuscular nutrition therapy formula based on a cost and an effectiveness of each of the food, the oral supplement, the topical treatment, the inhaled nutrition treatment, the intravenous nutrition therapy formula, and the intramuscular nutrition therapy formula.

9. The system of claim 8, wherein the server comprises multiple servers.

10. The system of claim 8, wherein at least one of the plurality of sensors is disposed within the electronic device.

11. The system of claim 8, wherein at least one of the plurality of sensors is disposed within a wearable device in communication with the electronic device.

12. The system of claim 8, wherein the electronic device is configured to receive a recommendation request from the individual.

13. The system of claim 8, wherein the system is configured to develop a nutrition plan based on the genetic information, medical information, therapeutic objectives, lifestyle information, and fitness data of the individual.

14. The system of claim 8, wherein the electronic device comprises a mobile device.

15. A method of creating a context-based personalized nutrition recommendation, the method comprising:
determining genetic information of an individual with a genetic test;
receiving medical information, therapeutic objectives, and lifestyle information of the individual;
mapping the genetic information, medical information, therapeutic objectives, and lifestyle information of the individual to one or more micronutrients;
identifying at least one of a food, an oral supplement, a topical treatment, an inhaled nutrition treatment, an intravenous nutrition therapy formula, and an intramuscular nutrition therapy formula that each provide the one or more micronutrients;
recommending the at least one of the food, the oral supplement, the topical treatment, the inhaled nutrition treatment, the intravenous nutrition therapy formula, and the intramuscular nutrition therapy formula based on a cost and an effectiveness of each of the at least one of the food, the oral supplement, the topical treatment, the inhaled nutrition treatment, the intravenous nutrition therapy formula, and the intramuscular nutrition therapy formula;
after the individual has received the one or more micronutrients via the recommended at least one of the food, the oral supplement, the topical treatment, the inhaled nutrition treatment, the intravenous nutrition therapy formula, and the intramuscular nutrition therapy formula, receiving feedback on an effectiveness of the recommended at least one of the food, the oral supplement, the topical treatment, the inhaled nutrition treatment, the intravenous nutrition therapy formula, and the intramuscular nutrition therapy formula; and
creating future recommendations for the individual and for other individuals based on the feedback.

16. The method of claim 15, wherein the lifestyle information comprises information about physical activities, mental activities, occupation, social network, financial security, culture, habits, and preferences of the individual.

17. The method of claim 15, further comprising receiving a recommendation request from the individual.

18. The method of claim 15, further comprising developing a nutrition plan based on the genetic information, medical information, therapeutic objectives, lifestyle information, and fitness data of the individual.

19. The method of claim 18, further comprising adjusting the nutrition plan based on the real-time air quality data associated with the location of the individual.

20. The method of claim 18, further comprising adjusting the nutrition plan based on updated medical information, therapeutic objectives, or lifestyle information of the individual.
